(19) United States
(12) Patent Application Publication
(10) Pub. No.: US 2022/0296141 A1
(43) Pub. Date: Sep. 22, 2022

(54) FLUID COLLECTION SYSTEMS AND METHODS AND COLLECTING FLUIDS

(71) Applicant: Patient Inventions, LLC, Scarsdale, NY (US)

(72) Inventors: Marc Richelsoph, Belmont, NC (US); Tamar Sobol, Scarsdale, NY (US); Noam Spanier, New York, NY (US)

(73) Assignee: Patient Inventions, LLC, Scarsdale, NY (US)

(21) Appl. No.: 17/805,524

(22) Filed: Jun. 6, 2022

Related U.S. Application Data
(63) Continuation of application No. PCT/US2020/063537, filed on Dec. 7, 2020.

(51) Int. Cl. A61B 5/20; A61M 25/00; A61M 39/02

(52) U.S. Cl. CPC A61B 5/208; A61M 25/0017; A61M 25/0097; A61M 39/02; A61M 2039/0202

(57) ABSTRACT

A fluid collection system for collecting fluid, such as urine through a series of valves and bags that isolate urine from the input to reduce risk of infection includes an input tube connected removably to a fluid supply tube, first and second valves separate from one another and each having flow-on and flow-off states and each fluidically connected to the input tube, respectively, to receive the input fluid and to output the input fluid when in the flow-on state, and at least one hollow collection container defining a water-tight interior and comprising a removable connector configured to removably connect to one of the first and second valves and, responsive to connection thereto, configured to receive the input fluid. The bags also contain a hydrogel to absorb the fluid. Input fluid pressure can be controlled and calibrated to maintain backpressure on the bladder.

### CROSS-REFERENCE TO RELATED APPLICATIONS

This is a continuing application, under 35 U.S.C. § 120, of copending international application No. PCT/US2020/063537, filed Dec. 7, 2020, which designated the United States and was published in English; this application also claims the priority, under 35 U.S.C. § 119, of U.S. Provisional Patent Application Nos. 62/944,601, filed Dec. 6, 2019 and 62/967,284, filed Jan. 29, 2020; the prior applications are herewith incorporated by reference in their entirety.

### STATEMENT REGARDING FEDERALLY SPONSORED RESEARCH OR DEVELOPMENT

Not Applicable

### FIELD OF THE INVENTION

The present systems, apparatuses, and methods lie generally in the field of bodily fluid collection bags. Specifically, the present disclosure relates to devices, systems, and methods that collect urine from urinary catheter systems.

### BACKGROUND OF THE INVENTION

Collection bags used for containing urine vary in size, shape, and construction. As can be seen in U.S. Pat. No. 6,887,230 to Kubalak, the collection bag is designed to be directly attached to a catheter and is part of the construction. Other versions have an input and output port. The input is separate from the catheter, which is connected to the input. The output allows for draining of the urine, such that the bag can be emptied and used over and over. As the risk of infection and contamination is possible, additional versions now have anti-reflux valves to reduce the amount of urine that can enter the catheter from the bag in certain situations.

Reusable bags, designed to be used multiple times, must be cleaned on a regular basis to reduce the risk of infection. The odor is strong and residual within the bag. Cleaning procedures recommend using a solution of vinegar and water to try and mitigate the odor. A reusable leg back requires using aseptic techniques, including hand hygiene and clean gloves. As the system is effectively open when the bag is removed for cleaning, the risk of infection increases.

Thus, a need exists to overcome the problems with the prior art systems, designs, and processes as discussed above.

Thus, a need exists to overcome the problems with the prior art systems, designs, and processes as discussed above.

### SUMMARY OF THE INVENTION

The present systems, apparatuses, and methods provide for a fluid collection system configured to reduce the risk of infection by maintaining a closed loop system to reduce exposure to bacteria and contaminants. A multiple-valve system isolates the fluid output and input to a bag, cartridge, or series of two bags or two cartridges. This allows for a relatively constant flow from the catheter to at least one of the input/output valve sets at all times and helps to reduce the risk of backflow while providing for constant washing of the output ports. In an exemplary embodiment, one output port is open at a time. However, the ports can be configured to allow both output ports to be open at the same time.

The fluid connection, coming from the catheter or other fluid source, in an exemplary embodiment, connects to a multiple valve array, which comprises two valves. The valves are linked to allow fluid to flow to each valve. The valves are initially closed, preventing fluid from leaking as well as bacteria or contaminants from entering the system. When one valve is opened, the fluid can flow from the fluid source or catheter through the valve into a bag or cartridge. With two valves connected, one can be open and the other closed, allowing one bag or cartridge to accept fluid. When the bag or cartridge is full, the open valve can be closed and the closed valve opened to allow fluid to flow into a second bag or cartridge.

While multiple types of valves can be used including manual valves, in an exemplary embodiment, the valves automatically open or close when a bag or cartridge is connected or removed.

In another exemplary embodiment, as the fluid enters the bag or cartridge, the fluid is absorbed by a hydrogel, turning the liquid into a semi-solid form. This action prevents any risk of leaking, positively locks the fluid into the hydrogel, and eliminates the sloshing of fluids. This change also assures that the bag or cartridge is disposable, eliminating the need to reuse and clean bags, while reducing the risk of infection.

Thus, the present systems, apparatuses, and methods provide a fluid collection system where the fluid input is connected to at least one valve that is initially closed.

The present systems, apparatuses, and methods provide a fluid collection system where the fluid input is connected to an array of at least two valves, both which are initially closed.

The present systems, apparatuses, and methods also provides for a fluid collection system where the fluid input is connected to an array of at least two valves, both which are initially closed, and the fluid input is the output flow from a catheter.

The present systems, apparatuses, and methods provide a fluid collection system where the fluid input is connected to at least one valve that is initially closed and, by connecting the valve to a bag or a cartridge, the valve is forced to open and allow fluid flow.

The present systems, apparatuses, and methods also provide a fluid collection system where the fluid input is connected to a multiple valve array whereby each valve is initially closed and, by connecting one of the valves to a bag or a cartridge, only the connected valve is forced to open and allow fluid flow.

The present systems, apparatuses, and methods also provides for a fluid collection system where the fluid input is connected to at least one valve which is initially closed and, by connecting the valve to a bag or a cartridge, the valve is forced to open and allow fluid flow. Disconnecting the bag or cartridge causes the valve to reseal and close.

The present systems, apparatuses, and methods provide a fluid collection system where the fluid input is connected to a multiple valve array in which each valve is initially closed and, by connecting either or both valves to a bag or a cartridge, only the connected valve or valves is/are forced to open and allow fluid flow.

The present systems, apparatuses, and methods provide a fluid collection system where the fluid input is connected to a multiple valve array in which each valve is initially closed. By connecting a bag or cartridge to the valve, the connected valve is forced into an open position to allow flow, while the additional valve(s) remain closed. Attaching an additional bag or cartridge to an additional valve forces the additional valve to open. Removing or disconnecting any bag or cartridge from any valve forces the valve to close.

The present systems, apparatuses, and methods provide a fluid collection system where the fluid input is connected to a multiple valve array in which each valve is initially closed and a bag or cartridge is connected to each valve, opening each connected valve. When fluid fills the first bag or cartridge to capacity, additional flow is directed to the next bag or cartridge without interruption.

The present systems, apparatuses, and methods provide a fluid collection system where the fluid input is connected to a multiple valve array in which each valve is initially closed and a bag or cartridge is connected to at least two valves, opening each connected valve. When fluid fills the first bag or cartridge to capacity, additional flow is directed to the next bag or cartridge without interruption and the full bag or cartridge can be removed, closing the connected valve and allowing a new empty bag or cartridge to be attached.

The present systems, apparatuses, and methods provide a fluid collection system where the fluid input is connected to a multiple valve array in which each valve is initially closed and bags or cartridges are connected to at least two valves, opening each connected valve. When fluid fills the first bag or cartridge to capacity, additional flow is directed to the next bag or cartridge without interruption and an indicator is triggered to notify the user that the first bag or cartridge is full and should be replaced.

The present systems, apparatuses, and methods provide a fluid collection system using bags or cartridges having at least one port.

The present systems, apparatuses, and methods provide a fluid collection system using bags or cartridges having at least one port and being partially filled with a fluid absorbing material. In an exemplary embodiment, the fluid absorbing material is one or more of or a combination of one or more of hydrogels, such as sodium polyacrylate, cellulose-based polyelectrolyte hydrogels, and other Super Absorbent Polymers (SAP).

The present systems, apparatuses, and methods provide a fluid collection system using bags or cartridges having at least one port and being partially filled with a fluid absorbing material. The fluid absorbing material expands to absorb at least some portion of the fluid entering the bag or cartridge.

The present systems, apparatuses, and methods provide a fluid collection system using bags or cartridges having at least one port and being partially filled with a fluid absorbing material. The fluid absorbing material expands to absorb all of the fluid entering the bag or cartridge until it is full, effectively locking the fluid into a semi-solid or solid mass.

The present systems, apparatuses, and methods provide a fluid collection system using bags or cartridges having at least one port and a valve in each port to prevent any leakage of fluid in the bag or cartridge from exiting the port.

The present systems, apparatuses, and methods provide a fluid collection system using bags or cartridges having at least one port and a valve in each port to allow fluid to enter while preventing any leakage of fluid in the bag or cartridge from exiting the port and a fluid absorbing material within the bag or cartridge.

The present systems, apparatuses, and methods provide for a fluid collection system using a first bag or cartridge having an inlet port and a valve that allows fluid to enter while preventing any leakage of fluid in the bag or cartridge from exiting the port and an exit port with valve that allows for fluid to flow to a second bag or connection once the fluid absorbing material within the bag or cartridge is saturated or full.

The present systems, apparatuses, and methods provide a fluid collection system using a first bag or cartridge in which the bag or cartridge remains in a collapsed state and expands as the fluid absorbing material expands.

The present systems, apparatuses, and methods provide a fluid collection system using a bags or cartridges in which at least two identical bags or cartridges are connected.

The present systems, apparatuses, and methods provide a fluid collection system using a bags or cartridges in which at least two dissimilar bags or cartridges are connected.

The present systems, apparatuses, and methods provide a fluid collection system having a first valve and a second valve such that engaging or locking the first valve to the second valve opens both valves, allowing fluid to flow through both valves.

The present systems, apparatuses, and methods provide a fluid collection system having a first valve and a second valve such that disengaging or unlocking the first valve from the second valve closes both valves, preventing fluid from flowing through either valve.

The present systems, apparatuses, and methods provide a fluid collection system using bags or cartridges in which the bag or cartridge is divided into two or more sections.

The present systems, apparatuses, and methods provide a system having a component requiring a minimum fluid pressure to allow fluid to flow into the bag or cartridge. Herein, “minimum fluid pressure” is defined as the minimum pressure required to cause at least partial filling of the bladder.

The present systems, apparatuses, and methods provide a system having a component in which the minimum fluid pressure to allow fluid to flow into the bag or cartridge is adjustable.

The present systems, apparatuses, and methods provide a bag or cartridge in which the bag or cartridge can expand as fluid enters, the expansion causing the bag or cartridge to expand or form into a given shape, for example, a polygon, a circle, a box, to name a few.

The present systems, apparatuses, and methods allow a separate tube to be attached directly to at least one of the valves to give a patient the ability to directly empty the fluid from inlet or catheter without the bag system.

The present systems, apparatuses, and methods allow an inline flow sensor to measure the fluid output from a catheter or fluid input.

The present systems, apparatuses, and methods allow an inline flow sensor to measure the fluid output from a catheter or fluid input and digitally record the flow.

The present systems, apparatuses, and methods allow an inline flow sensor to measure the fluid output from a catheter or fluid input and digitally record the flow where low and or high volumes trigger a warning to the patient.

The present systems, apparatuses, and methods allow an inline flow sensor to measure the fluid output from a catheter or fluid input and digitally record the flow and transit such data wirelessly to a recorder, receiver, or remote health care monitoring device or facility, for example, through an internet connection.

The present systems, apparatuses, and methods allow an inline flow sensor to measure the fluid output from a catheter or fluid input, a device to measure fluid volume, and provide the ability to record or transmit such data wirelessly to a recorder, receiver, or remote health care monitoring device or facility.

The present systems, apparatuses, and methods allow a sensor to be place along the input tube, or other portion of the assembly, such that the sensor can detect color or color changes from the input.

The present systems, apparatuses, and methods allow a self-contained unit to sense changes in fluid volume over time.

The present systems, apparatuses, and methods allow a self-contained unit to sense changes in fluid volume over time by calculating weight of the fluid.

The present systems, apparatuses, and methods allow a self-contained unit to sense changes in fluid volume over time by calculating weight of the fluid and converting the weight to volume.

The present systems, apparatuses, and methods allow a self-contained unit that senses changes in fluid volume over time and transmits the data received to a remote receiver.

The present systems, apparatuses, and methods allow a self-contained unit having an opening to receive at least one cartridge or bag that, when full, can be removed.

The present systems, apparatuses, and methods allow a self-contained unit having an opening to receive at least one cartridge or bag that, when full, can be removed while flow continues to a second cartridge or bag.

The present systems, apparatuses, and methods provide a self-contained unit having an opening, a liner, and at least one cartridge or bag.

The present systems, apparatuses, and methods provide a unit having a receiver accepting fluid and an electronic measuring device measuring weight or volume of the incoming fluid.

The present systems, apparatuses, and methods provide a unit having a receiver accepting fluid and an electronic measuring device measuring the weight or volume of the incoming fluid and supplying the measured data in a readable form, for example, as charts, graphs, points, or other desired data digitally to a display on the unit and/or to a remote display.

With the foregoing and other objects in view, there is provided, a fluid collection system comprising a hollow tube comprising a tube input configured to connect removably to a fluid supply tube and, responsive to connection, to receive input fluid therethrough, and a tube output configured to output the input fluid, a first valve having flow-on and flow-off states and comprising a first input configured to regulate an amount of flow through the first valve and a first output, a second valve having flow-on and flow-off states and comprising a second input configured to regulate an amount of flow through the second valve and a second output, the first and second valves each fluidically connected to the tube output, configured to receive the input fluid respectively, and configured to output the input fluid from the hollow tube when in the flow-on state, and at least one collection container comprising a fluid bag defining a water-tight interior and comprising an input shaped to receive fluid therethrough and into the interior and a removable connector comprising an input configured to removably connect to one of the first and second outputs, comprising an output fixed to the fluid bag and defining a lumen fluidically connected to the interior of the fluid bag, and, responsive to connection to the one of the first and second outputs, configured to receive fluid from the hollow tube through the one of the first and second outputs.

With the objects in view, there is also provided a fluid collection system comprising a hollow input tube configured to connect removably to a fluid supply tube supplying input fluid, first and second valves separate from one another and each having flow-on and flow-off states, each of the first and second valves fluidically connected to the hollow input tube, respectively, to receive the input fluid and to output the input fluid when in the flow-on state, and at least one hollow collection container defining a water-tight interior and comprising a removable connector configured to removably connect to one of the first and second valves and, responsive to connection thereto, configured to receive the input fluid.

With the objects in view, there is also provided a fluid collection system comprising a hollow tube comprising a tube input configured to connect removably to a fluid supply tube and, responsive to connection, to receive input fluid therethrough and a tube output configured to output the input fluid, a valve having flow-on and flow-off states and comprising a valve input configured to receive fluid flow therethrough and at least two outputs including a first output and a second output. The valve separates the valve input from the at least two outputs and directs flow independently from the valve input to each of the at least two outputs.

With the objects in view, there is also provided a pressure control valve fluidically connected to the tube input, having a pressure flow setting, and being configured to permit flow of the input fluid into the tube input when the input fluid is at a pressure above the pressure flow setting.

In accordance with another feature, the first and second valves have a steady state in the flow-off state and connection to the at least one collection container automatically places either of the first and second valves in the flow-on state and disconnection from the at least one collection container automatically returns the one of the first and second valves to the flow-off state.

In accordance with a further feature, the at least one collection container is a modular set of at least first and second collection containers each respectively connected to one of the first and second outputs to permit one of the at least first and second collection containers to be in either of the flow-on and flow-off states while the other of the at least first and second collection containers are in either of the flow-on and flow-off states.

In accordance with an added feature, the at least first and second collection containers are configured such that, responsive to the interior of one of the at least first and second collection containers being full, flow of the input fluid is directed to the other of the at least first and second collection containers.

In accordance with an additional feature, the removable connector of the at least one collection container has a container valve configured to enter a fluid-closed state automatically when the interior is full of liquid.

In accordance with yet another feature, there is provided a fluid monitoring system connected to the hollow tube and configured to measure fluid output and to transmit data associated with the fluid output to a receiver to permit direct reading of the fluid output.

In accordance with yet a further feature, the fluid monitoring system has a defined measurement range for the fluid output and is configured to indicate if the measured fluid output falls at least one of within the defined measurement range and outside the defined measurement range.

In accordance with yet an added feature, the fluid monitoring system is configured to transmit and remotely create an alarm if the measured fluid output is outside the defined measurement range.

In accordance with yet an additional feature, there is provided a fluid monitoring system connected to the hollow tube and configured to measure at least one of fluid color and fluid transparency and to transmit data associated with the measurement of the at least one of fluid color and fluid transparency to a receiver to permit direct reading of the at least one of fluid color and fluid transparency.

In accordance with again another feature, the fluid monitoring system has a defined measurement range for the at least one of fluid color and fluid transparency and is configured to indicate if the at least one of the measured fluid color and the measured fluid transparency falls at least one of within the defined measurement range and outside the defined measurement range.

In accordance with again a further feature, the fluid monitoring system is configured to transmit and remotely create an alarm if at least one of the measured fluid color and measured fluid transparency is outside the defined measurement range.

In accordance with again an added feature, there is provided a base unit comprising a scale configured to read a weight of the at least one collection container and a container section shaped to contain thereon the at least one collection container and position the at least one collection container on the scale to read the weight of the at least one collection container.

In accordance with again an additional feature, the base unit comprises a monitoring system configured to transmit at least the weight to a remote receiver and permit direct reading of the weight.

In accordance with still another feature, the remote receiver is a remote computing transceiver connected to the Internet and configured to receive information from and transmit information to the base unit.

In accordance with still a further feature, the base unit comprises a monitoring system configured to display measured data to a user at least one of visually, audibly, and tactily.

In accordance with still an added feature, there is provided a hospital bed connector configured to secure at least one of the hollow tube, the first and second valves, and the at least one collection container to a hospital bed.

In accordance with still an additional feature, the at least one collection container comprises a quantity of fluid-absorbing media within the interior.

In accordance with a concomitant feature, the interior of the at least one collection container is subdivided into at least two fluid-receiving compartments.

Although the systems, apparatuses, and methods are illustrated and described herein as embodied in fluid collection systems and methods for collecting fluids, it is, nevertheless, not intended to be limited to the details shown because various modifications and structural changes may be made therein without departing from the spirit of the invention and within the scope and range of equivalents of the claims. Additionally, well-known elements of exemplary embodiments will not be described in detail or will be omitted so as not to obscure the relevant details of the systems, apparatuses, and methods.

Additional advantages and other features characteristic of the systems, apparatuses, and methods will be set forth in the detailed description that follows and may be apparent from the detailed description or may be learned by practice of exemplary embodiments. Still other advantages of the systems, apparatuses, and methods may be realized by any of the instrumentalities, methods, or combinations particularly pointed out in the claims.

Other features that are considered as characteristic for the systems, apparatuses, and methods are set forth in the appended claims. As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one of ordinary skill in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the systems, apparatuses, and methods of the invention that are regarded as novel, it is believed that the systems, apparatuses, and methods will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the systems, apparatuses, and methods. Advantages of embodiments of the systems, apparatuses, and methods will be apparent from the following detailed description of the exemplary embodiments thereof, which description should be considered in conjunction with the accompanying drawings in which:

FIG. 1 is a fragmentary, perspective view of an exemplary embodiment of a fluid collection system having a single valve and connection;

FIG. 2 is a fragmentary, perspective view of an exemplary embodiment of a fluid collection system having a multi-valve array;

FIG. 3 is a fragmentary, perspective view of an exemplary embodiment of a fluid collection system having a multi-valve array with and input pressure valve;

FIG. 4 is a fragmentary, perspective and partially cut-away view of the fluid collection system of FIGS. 2 and 3 with the cut-away in a section of the valve array;

FIG. 5 is a cross-sectional view of an exemplary embodiment of a pressure valve;

FIG. 6 is a perspective view of an exemplary embodiment of a fluid retention bag and input connector;

FIG. 7 is a fragmentary, perspective view of the fluid retention bag and input connector of FIG. 6 with the multi-valve array of FIGS. 2 and 3 in a disconnected state;

FIG. 8 is a fragmentary, perspective view of the fluid retention bag and input connector of FIG. 6 with the multi-valve array of FIGS. 2 and 3 in a connected state of the bag to one of the valves;

FIG. 9 is a fragmentary, perspective view of two of the fluid retention bags and input connectors of FIG. 6 with the multi-valve array of FIG. 3 in a parallel connected state;

FIG. 10 is a fragmentary, perspective view of an exemplary embodiment of a multi-compartment fluid retention bag from above a side thereof;

FIG. 11 is a perspective view of the multi-compartment fluid retention bag of FIG. 10 from above;

FIG. 12 is a fragmentary, perspective view of an exemplary embodiment of a multi-bag and multi-valve assembly;

FIG. 13 is a fragmentary, perspective view of the multi-bag and multi-valve assembly with an exemplary embodiment of an assembly container;

FIG. 14 is a fragmentary, perspective view of the multi-bag and multi-valve assembly and container of FIG. 13 from a side thereof;

FIG. 15 is a fragmentary, perspective view of an exemplary embodiment of a cartridge- or bag-based fluid-collection unit having a base and container;

FIG. 16 is a perspective, exploded view of the base and container of the unit of FIG. 15;

FIG. 17 is a perspective, exploded view of the base and container of the unit of FIG. 15 assembled and an exemplary embodiment of a liner; and

FIG. 18 is a perspective view of the cartridge of the unit of FIG. 15.

### DETAILED DESCRIPTION OF THE EMBODIMENTS

As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one skilled in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the features of the systems, apparatuses, and methods that are regarded as novel, it is believed that the systems, apparatuses, and methods will be better understood from a consideration of the following description in conjunction with the drawing figures, in which like reference numerals are carried forward.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Alternate embodiments may be devised without departing from the spirit or the scope of the invention. Additionally, well-known elements of exemplary embodiments of the systems, apparatuses, and methods will not be described in detail or will be omitted so as not to obscure the relevant details of the systems, apparatuses, and methods.

Before the systems, apparatuses, and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

It will be appreciated that embodiments of the systems, apparatuses, and methods described herein may be comprised of one or more conventional processors and unique stored program instructions that control the one or more processors to implement, in conjunction with certain non-processor circuits and other elements, some, most, or all of the functions of the systems, apparatuses, and methods described herein. The non-processor circuits may include, but are not limited to, signal drivers, clock circuits, power source circuits, and user input and output elements. Alternatively, some or all functions could be implemented by a state machine that has no stored program instructions, or in one or more application specific integrated circuits (ASICs) or field-programmable gate arrays (FPGA), in which each function or some combinations of certain of the functions are implemented as custom logic. Of course, a combination of these approaches could also be used. Thus, methods and means for these functions have been described herein.

The terms “program,” “software,” “software application,” and the like as used herein, are defined as a sequence of instructions designed for execution on a computer system or programmable device. A “program,” “software,” “application,” “computer program,” or “software application” may include a subroutine, a function, a procedure, an object method, an object implementation, an executable application, an applet, a servlet, a source code, an object code, any computer language logic, a shared library/dynamic load library and/or other sequence of instructions designed for execution on a computer system.

Herein various embodiments of the systems, apparatuses, and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

There is provided a new system for fluid collection which uses valves that control fluid flow and, in an exemplary embodiment, at least two collection bags. While bags can be reusable, it is preferred that they be disposable. When connected to the valve(s), the valves and disposable bags create a close loop system to isolate and contain fluid flow, germs, and contaminants from entering the system. The system is designed to be connected to a catheter or other tube that allows for fluid flow to enter.

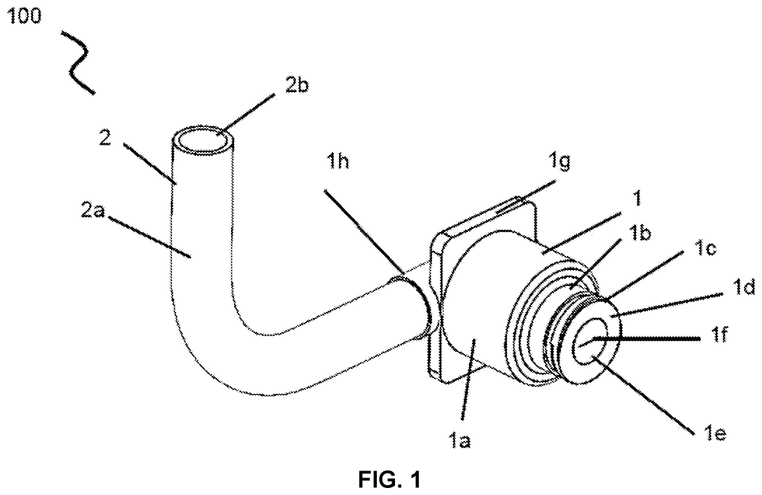

Described now are exemplary embodiments of the present systems, apparatuses, and methods. Referring now to the figures of the drawings in detail, there is shown a first embodiment of a fluid collection system, illustrated generally at 100, as shown in FIG. 1. A valve 1 comprises an external case 1a, an extension 1b having an external thread 1c, a front surface 1d, an internal component 1i having a face 1e that can be pushed or compressed inwards, and an opening 1r, e.g., a slot. When the internal component face 1e is compressed or pushed inwards, opening 1f opens to allow for fluid to flow through the valve 1. When the internal component face 1e is at rest (i.e., in a resting position or a steady state), fluid cannot pass through the opening 1f. This configuration is an exemplary embodiment of a valve that automatically stops fluid flow when the internal component face 1e is in a resting position. A back plate 1g and rear tube 1h are part of this valve embodiment and can vary depending on the valve used. An input tube 2 with an outer wall 2a and bore 2b can be a separate tube from the catheter or an input tube, or it can be the actual end of the catheter or the input tube.

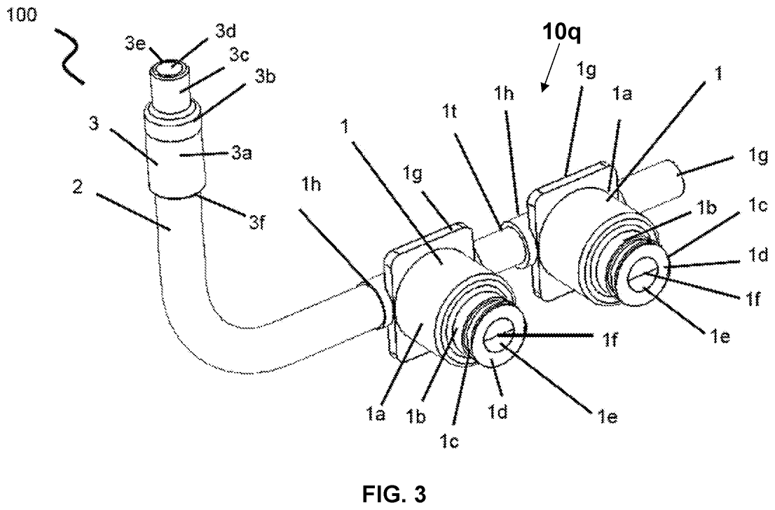

As shown in FIG. 3, the embodiment 100 of the fluid retention system generally comprises an array of the valve 1 along with a pressure control/inducing valve 3. In a normal catheter and bag construct, fluid from the catheter flows directly into the bag, thereby maintaining a relatively zero pressure model whereby the bladder is always as empty as possible. By providing a pressure control or pressure inducing valve 3, the pressure required to allow fluid drainage into the bag can be set or controlled. The pressure inducing valve 3 has a pressure flow setting, thereby permitting flow when the input fluid is at a pressure above the pressure flow setting. By allowing fluid pressure to build up within the bladder to a reasonable user-selectable level before the valve 3 opens, the bladder can be exercised to maintain flexibility and usefulness. In this exemplary configuration, the valve 3 comprises a main body 3a, an upper portion 3b, a input tubing connection portion 3c, an internal bore 3d extending into the valve 3 from upper face 3e, and a lower face 3f, which faces in a direction of the array of valves 1. To clarify that more than two valves 1 can be attached to create a valve assembly/assemblies 10q can be attached together, input tube 1t is shown. Input tube 1g is a conduit to which another valve 1 can be connected. Of course, in a construct of one of the valves 1, the input tube 1t is plugged or is simply absent and the valve 1 is sealed at the beginning of 1g.

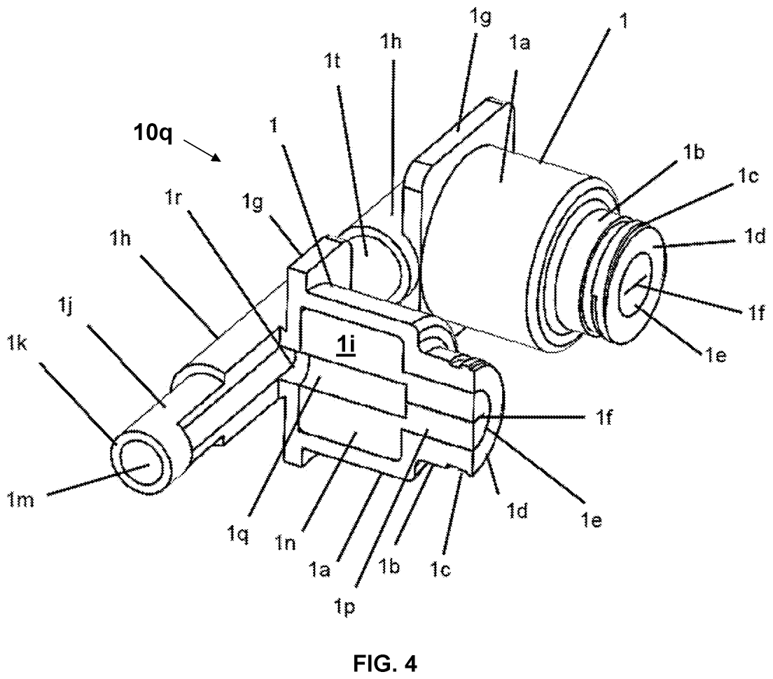

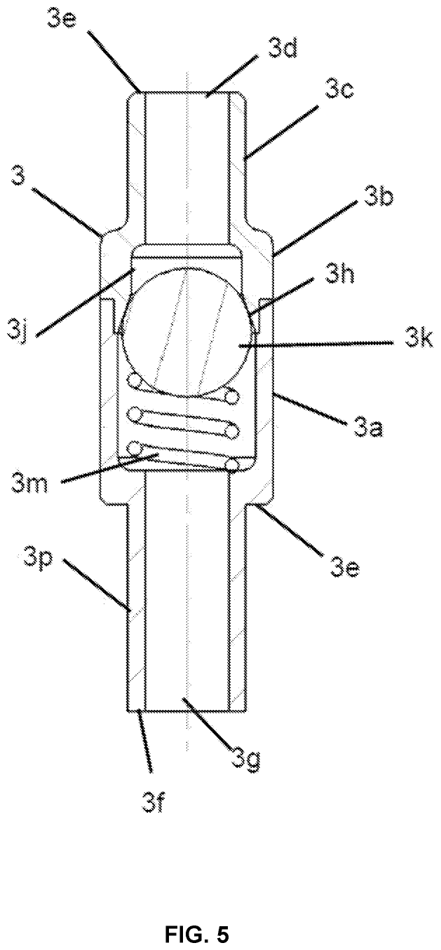

FIG. 4 shows the detail of an exemplary embodiment of a valve 1. Of course, other types of valves can be used. In this example, the external portion of the valve 1 is constructed from a hard polymer and the internal component 1i or insert is constructed from a soft elastomer, such as silicone. The internal component face 1e is elastomeric and compressible. By compressing the front face 1e inwards, the slot if is forced open to allow fluid flow through the valve 1. The cross-sectioned part of FIG. 4 shows that fluid flows from the opening 1m through to a bore 1q in the elastomeric valve section of the internal component 1i, and finally into the slot if when in an open position. The valve 1 has a tubular section 1j with a front face 1k, to which tubing is connected, and a bore 1m. The elastomeric portion is always sealed and closed until face 1e is forced inwards. Such a configuration prevents fluid from leaking out and germs and other contaminants from entering in. FIG. 4 shows two valves 1 connected by the input tube 1t, which can be molded as part of one of the valves or the input tube 1t can be a separate lumen. Of course, there are other examples of valves that automatically open and close, including those similar to the valve shown in FIG. 5, that use a spring or other types of valves.

FIG. 5 shows a section view of an exemplar embodiment of the pressure control or pressure inducing valve 3. A top or upper portion 3b is heat sealed, or adhesively bonded to a lower section 3g of the main body 3a, creating a sealed unit. A ball 3k contacts a seat 3h when the ball 3k is in the resting, zero-pressure configuration (e.g., the steady state). The seat 3h connects to a bore 3j to allow the ball 3k to fully seat against the seat 3h. A spring 3m, which can be an elastomer or metallic spring, applies pressure against the ball 3k to close off flow in the steady state. An amount of pressure needed to compress the ball 3k against the seat 3h can be altered by changing a spring rate of the spring 3m. Thus, pressure in the bladder is force to reach a certain desired level before the valve 3 will open and allow fluid flow to pass through the system and into collection bags. It is noted that there are other types of valves that can stop flow and this valve 3 is only an exemplary embodiment. It is also possible to use a valve that can be opened or actuated by an external, non-illustrated controller. Thus, the valve 3 can be electric or magnetic, and controlled by a control system that monitors pressure and/or it can be opened manually by the user at a desired time.

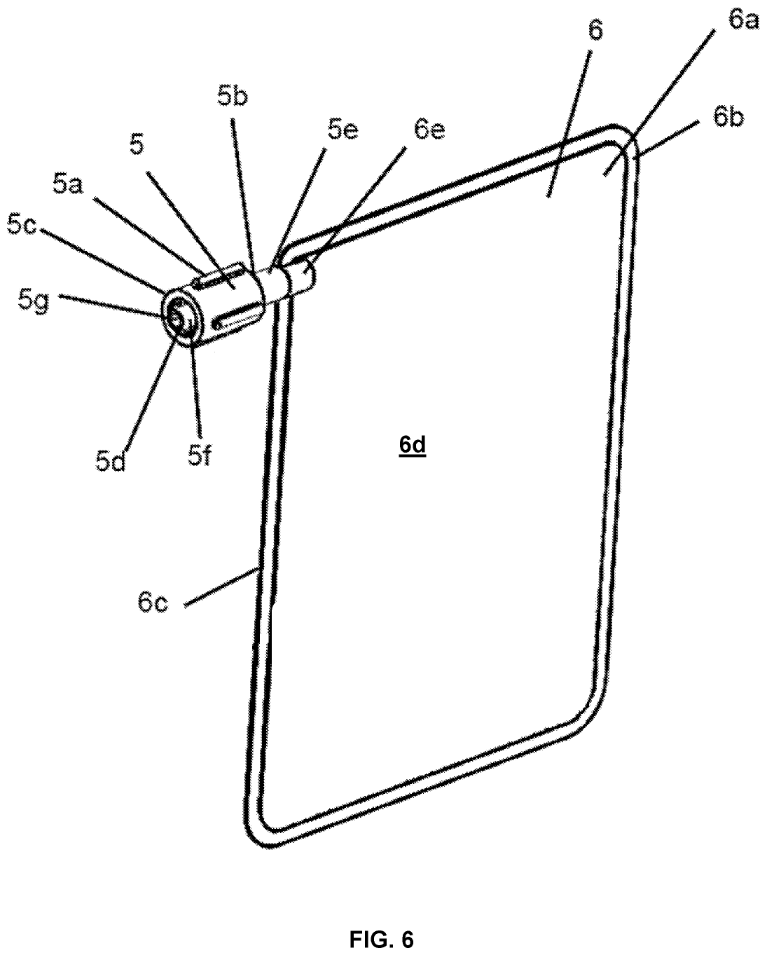

A fluid collection container 6, for example, a bag, with a connector 5 is shown in FIG. 6. The connector 5 comprises a rotational component 5a having a back face 5b and a front face 5c, an internal portion having a front face 5d and rear tubular section 5e, and a nose 5f extending inwards from front face 5d. The rotational component 5a has internal threads that engage corresponding threads of the valve 1 when connected and turned (e.g., as a Luer connector). The fluid collection bag 6 has a section 6a that is formed by a heat seat or seal 6b of two side pieces 6c, 6d of flexible polymer, such that section 6a becomes a hollow bag that can be inflated or deflated, in particular, the bag can expand as it is filled with fluid. The rear section 5e of the connector 5 is sealed to an opening 6e in the fluid collection bag 6, which opening 6e can be in the heat seal 6b or in one of the side pieces 6c, 6d.

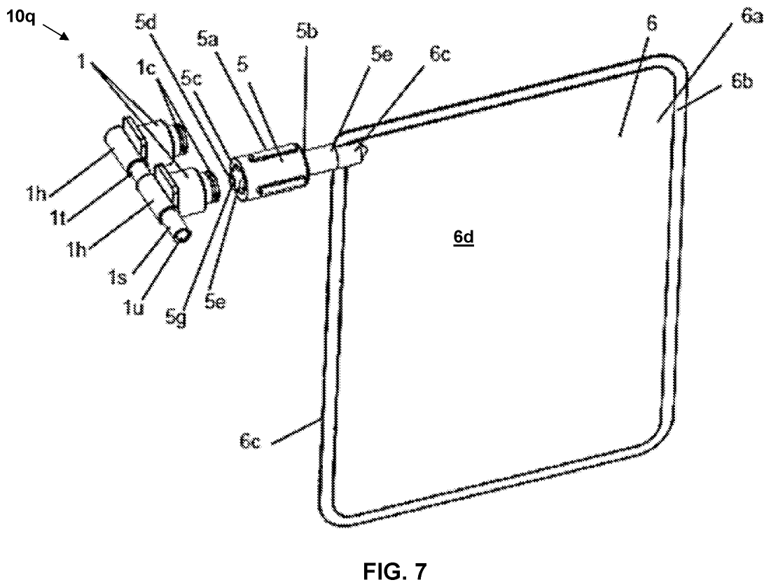

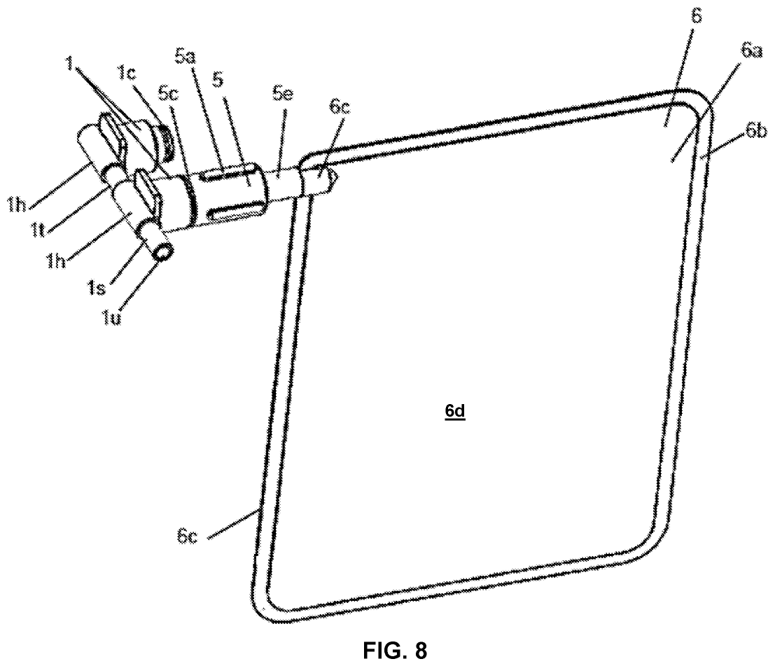

In FIGS. 7 and 8, a two valve assembly 10q can be seen with a fluid collection bag and connector assembly. By lining up the connector with the valve, the end 5d of nose 5e presses against 1e of valve 1. When rotational component 5a is turned clockwise, end 5d is driven deeper into the valve, forcing the slot if to open. The second valve, as it is not connected to a fluid collection bag and connector is still in the closed position.

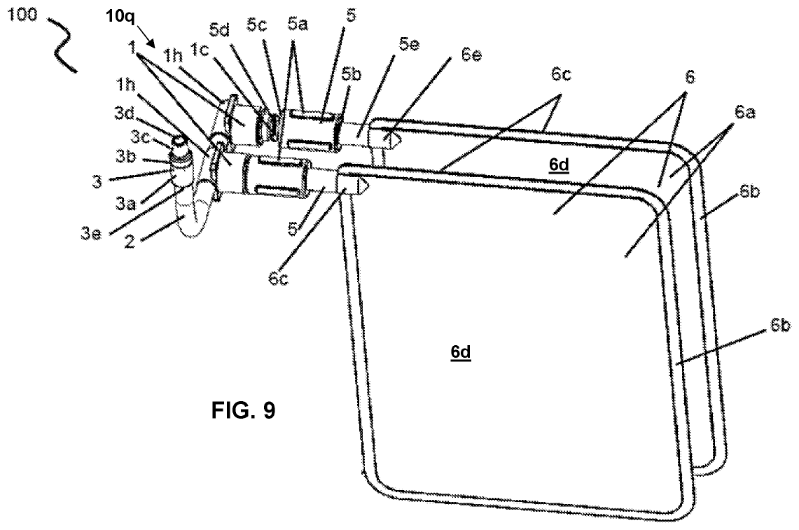

FIG. 9 shows two fluid collection bags 6 with a first fluid connection bag 6 connected to an upstream valve 1 and a second fluid collection bag 6 having a connector 5 in a position to be connected to a downstream valve 1 of a valve assembly 10q. Once connected, fluid can flow into the second fluid collection bag 6. A configuration of a multiple valve assembly 10q (with two or more valves 1) and a set of fluid collection bags 6 (with two or more bags 6) allows for any number of different combinations for a fluid collection system 100. In such a configuration, operation of the valves 1 also have a large number of operating processes. For example, if the first valve 1 is closed with no bag 6 attached, the second valve 1 can be either closed or in an open position with the connector 5 of a fluid bag 6 attached to the second valve 1. Likewise, both valves 1 can be open with two collection valves 1 and connectors 5 attached thereto. Such a configuration allows for the user to be able to use one bag 6 at a time, or to use two bags 6 at the same time for increased volume capacity, or to use two bags 6 in series to allow for overflow from a full first bag 1 to flow into the empty second bag 6. There is no restriction on the number of valves 1 or fluid bags 6 possible in such an exemplary array. This expandability, of course, is limited by the practicality of mobile applications, where the bag 6 or bags 6 are worn by the user, therefore, limiting the actual number of bags 6 that can be used at the same time.

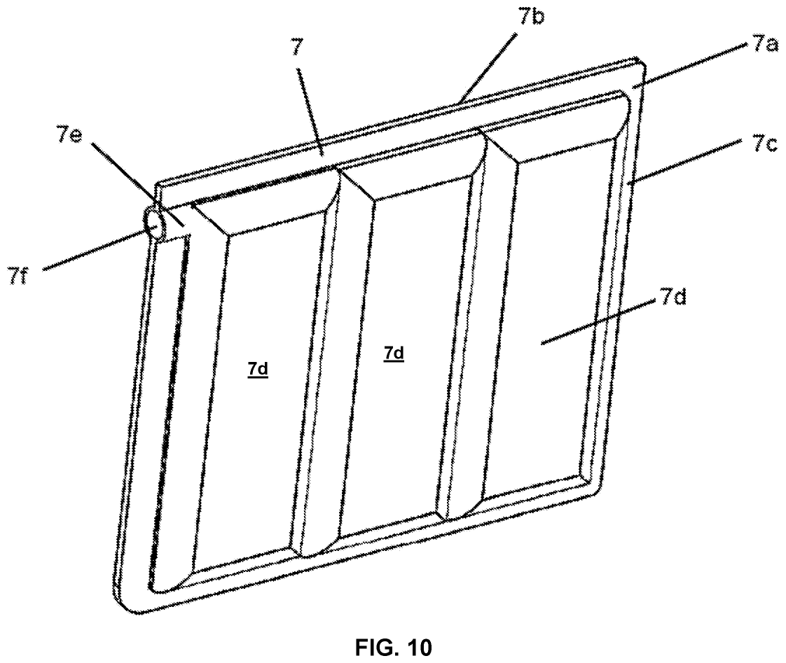

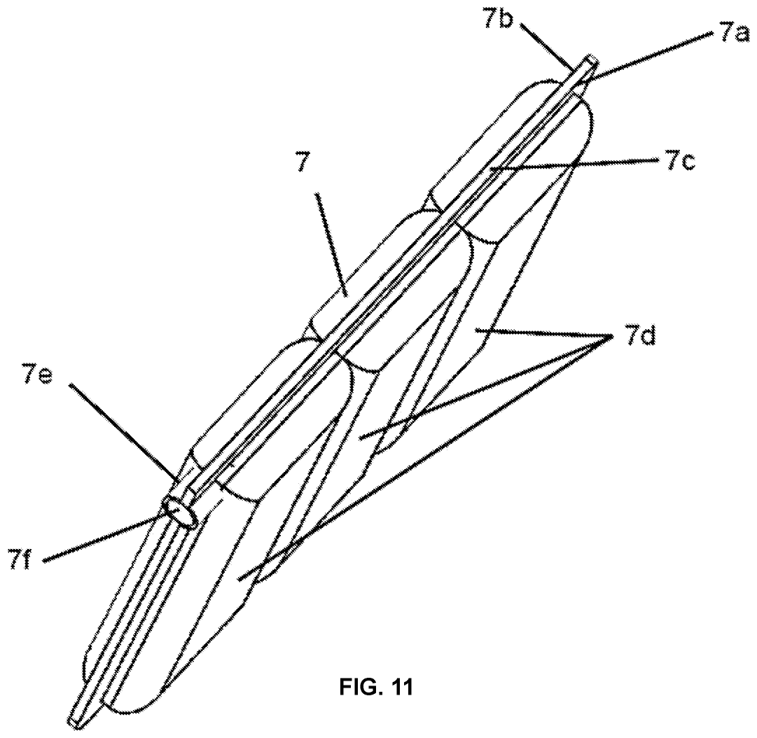

FIGS. 10 and 11 illustrate another exemplary embodiment of a fluid collection device as a multi-chamber bag 7 in a different configuration than the fluid collection bag 6. Rather than having a single fluid collecting chamber, the bag 7 is divided into two or more chambers 7d. The bag 7 comprises two sheets (e.g., of a polymer), a front sheet 7a and back sheet 7b, which are sealed together at a seal 7c. The chambers 7d are formed by additional heat sealing or with an adhesive or other fastening process or method. A generally cylindrical section 7e and an opening 7f is formed between the sheets 7a, 7b to permit attachment of the bag 7 to a tubing connector (e.g., the connector 5). An advantage of a multi-chamber bag 7 is that the chambers 7d help to control outward expansion of the bag 7, keeping it thinner overall as the bag 7 fills.

In a desirable exemplary configuration, the fluid collection bags 6, 7, and cartridge 20 described herein contain an amount of hydrogel to absorb and lock in fluid as it enters through the system 100 into the bag 6, 7. The hydrogel turns the fluid into a gel. Such a conversion prevents fluid from backing up into the valve 1, thereby further reducing any risk of contamination across the valve 1, and also preventing any possibility of leakage of fluid from the bag 6, 7 (e.g., in the event that all the fluid in the bag 6, 7 is absorbed by the hydrogel). It is also desirable to have bags 6, 7, and the hydrogel be resorbable so that both dissolve over time to reduce land fill waste.

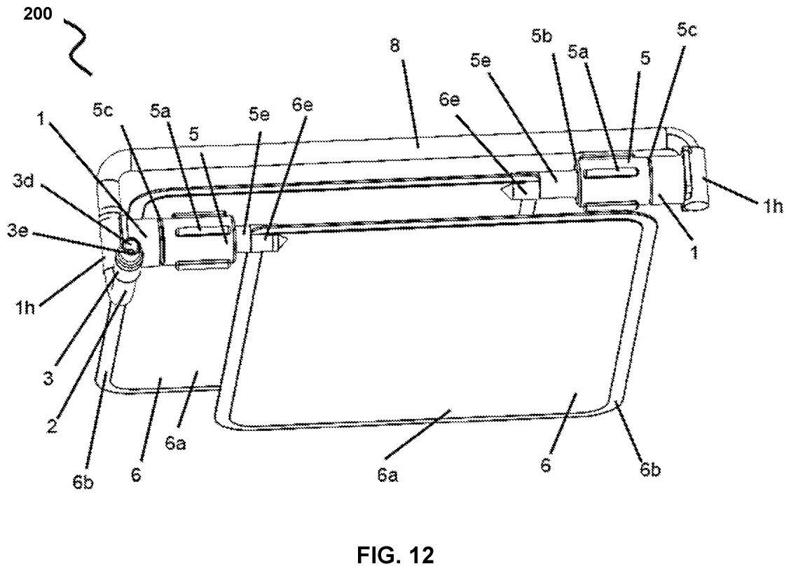

FIG. 12 shows another exemplary embodiment of a fluid collection system 200 having the basic components described herein. This configuration shows how the components can be rearranged to achieve a similar result. Rather than having the valves 1 connected side-by-side, the valves 1 are configured to face in opposing directions. The valves 1 are connected in series by a connection tube 8 that is longer than the distance between the valves in the system 100. The configuration spreads the valves 1 apart and allows a user to have easier access to the connectors 5 so that they can be turned by hand with less interference. It is also a compact configuration that is modular and/or repeatable in an advantageous way.

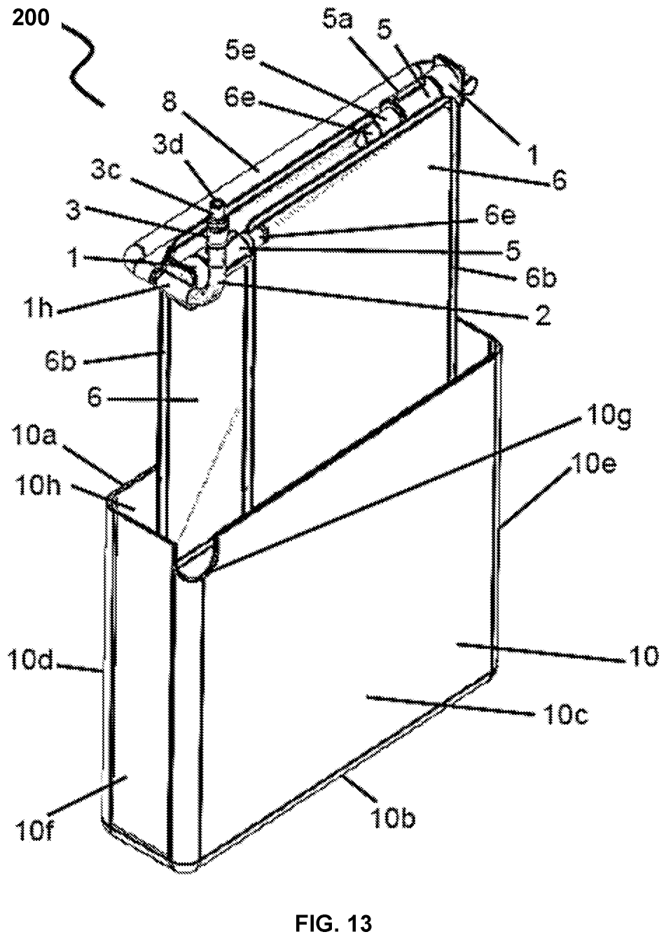

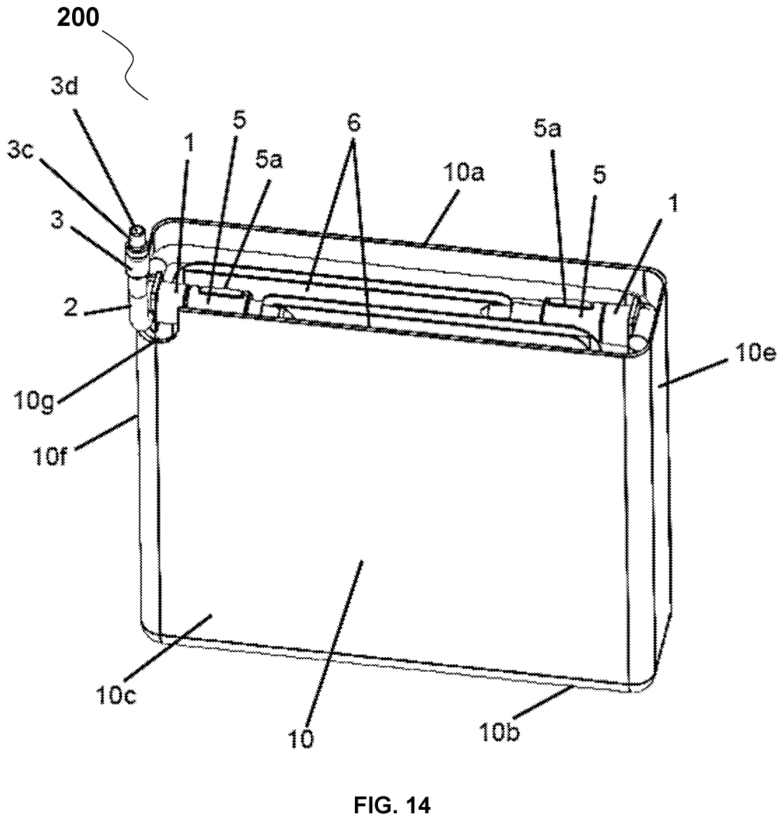

In particular, shown in FIGS. 13 and 14 is an exemplary configuration of the fluid collection system 200 in a holder or case 10. The holder 10 has an upper surface 10a, a lower surface 10b, a front face 10c, a back face 10d opposite the front face 10c, a first side face 10e, a second side face 10f opposite the first side face 10e, and a cut out 10g to accommodate the input tube 2. The various surfaces of the holder 10 defines an internal pocket 10h shaped to receive at least one fluid collection system 200 therein. The holder 10 can be shaped and sized to accommodate more than one of the fluid collection systems 200 in a modular configuration that makes replacement of each fluid collection system 200 easy to remove and replace. While the case 10 can be constructed from rigid materials, for example, for use when fixed to a structure such as a wheelchair or bed, the case 10 can also be made softer and/or flexible, such as in the shape of a purse for carrying by a user. Exemplary materials for a user-portable case 10 include natural fibers, such as cotton, hemp, flax, or silk, or flexible polymeric materials, such as polyester, polypropylene, polyethylene, and nylon, to name a few of each.

In the exemplary embodiments, the connector 5 can also be replaced with another non-illustrated valve, such as a ball valve. This second valve, as it cooperates with the valve 1 connected to the bag 6, 7, is also moved into an open/drain position. Thus, the second valve provides other measures for stopping leakage from the fluid retention bag 6, 7, as well as an additional way of reducing the risk of backflow and potential contamination of the system 100, 200.

By creating a multi-valve assembly for the systems 100, 200, 300, there are numerous benefits. The valves 1 are relatively small and inexpensive and add in an ability to have a main input from a urinary catheter and to provide disposable fluid-retention bags that can be easily replaced while reducing any risk of contamination. The valves described herein automatically close, in other words, removal of a bag 6, 7, immediately cuts off fluid flow by entering into a fluid-closed state, thereby eliminating any risk of back flow, which can carry contaminants or bacteria. In a particularly desirable embodiment, the valves 1 are also swab-able and/or able to be easily wiped down with alcohol between changes of the bag(s) 6, 7 to further reduce any risk of contamination.

A multiple bag configuration also allows for more than two bags 6, 7 to be included in the system 100, 200, 300. In such a case, fluid flow can be directed to fill both bags if two are attached, for example, or the flow can be directed to the first bag until it is full and then into the second by setting one of the valves 1 at a higher flow start pressure than the other. Rather than having to take a reusable bag and clean it, the systems 100, 200, 300 provide an easy way to disconnect a full bag 6, 7, dispose of the bag 6, 7, and connect a new clean and sterile bag 6, 7 to the valve 1. This makes the system 100, 200, 300 easy to use and extremely sanitary over the prior art. By including a hydrogel in the bag 6, 7, any fluid is locked into place within the gel. The fluid is converted from omnidirectional movement into a substantially stable solid that is not able to slosh around and leak. In another particularly desirable configuration, providing the bag 6, 7 and the hydrogel as resorbable or compostable materials, the large amount of such waste reduces adverse environmental impact

As discussed, a pressure valve 3 allows a user to control input pressure to an array or assembly of valves and can be set for a minimum level. In certain circumstances, allowing the bladder to drain without resistance can weaken the bladder's ability to function after long-term catheter usage. Therefore, the valve 1 can be of a type that is activated by the user, such as a squeezing valve that opens under compression, a magnetically activated valve that opens by applying a magnet, or an automatically actuated valve with a motor or solenoid that uses electrical current. Using an electrically based system allows for feedback to be supplied to the user for circumstances when the bladder should be emptied, for example, by measuring pressure in the valve 1, or when the bags 6, 7 should be emptied, for example, by providing an electronic scale or measuring instrument within the case 10.

To further allow a patient to control emptying of the bladder, the user can bypass the bag system 100, 200, 300 and directly attach a tube to at least one valve 1 of the valve assembly 10q. Such a use opens the valve 1 and allows direct flow of fluid out of the system 100, 200, 300. This is helpful in certain situations, such as using feedback from the system 100, 200, 300 that notifies the patient that an input pressure is at a level sufficient to require emptying of the bladder, where facilities are available to allow direct emptying of the bladder without the need to use one of the bags 6, 7.

In addition to measuring pressure, or as a separate alternative, it is beneficial to monitor flow of urine or liquid through the system 100, 200, 300. A lack of sufficient flow may indicate additional health problems that may need immediate treatment. In addition, urine output and tracking may be beneficial information for the physician in treating a patient. A flow meter can be placed in-line with input tube 2, for example, and the data collected digitally. This data can be collected by an onboard computing and/or storage device and/or transferred via wire or wireless data transfer to an external receiver, for example, over the Internet. The data can then be provided to the physician, whether manually or automatically. In a situation where fluid flow is either approaching or at a dangerous level, the computer can issue a warning to the patient or provide notification to the health care provider. Rather than have someone checking the data at intervals, automatic, periodic examination of the data is a safer approach, as feedback to the health care provider and the patient is immediate.

Flow sensors to measure flow rate are well understood in the art. Such sensors use paddle wheels or other mechanical components that turn under the pressure of the flow and provide data output. Other sensors are entirely digitally, for example, where the sensors detect heat differences over a length of fluid to determine the rate of flow. Optical fluid flow sensor options are available and are envisioned as well.

Coupling flow sensor data with weight measurement of the bags provides volume and flow rate data. For patients with compromised kidney or bladder disease or cancer, such information provides helpful detailed information to a medical professional. In addition, any changes to a baseline could be noted in real-time and allow for faster treatment or treatment changes.

While the monitoring systems described herein can be mobile for use by the patient outside the hospital, it is also beneficial for hospital staff, especially with critical care patients. In such a circumstance, data from the systems is transmitted directly to a monitoring station that provides status alerts. Rather than checking on a patient's urinary output flow at large time intervals, the system is monitored remotely and automatically. Any dangerous levels sends an immediate warning to the health care providers.

In an additional exemplary embodiment, the monitoring system adds an additional sensor or sensors to detect color, color changes, or transparency of the fluid entering the system. By placing a photovoltaic cell on one side of a tube or other section of the system 100, 200, 300 and by placing a light source on the other side of the cell, color changes can be detected, such as blood entering the tube and reducing light transmission. Photovoltaic cells output a voltage that can be monitored. Changes in the voltage are used to indicate normal or abnormal light transmission. This technique is inexpensive and easily incorporated into the systems 100, 200, 300 herein. In one exemplary camera embodiment, a charge-coupled device (CCD) or CMOS detector can be used. By using a camera as the detection device, the exact color can be charted and changes noted more completely than a simple photovoltaic cell.

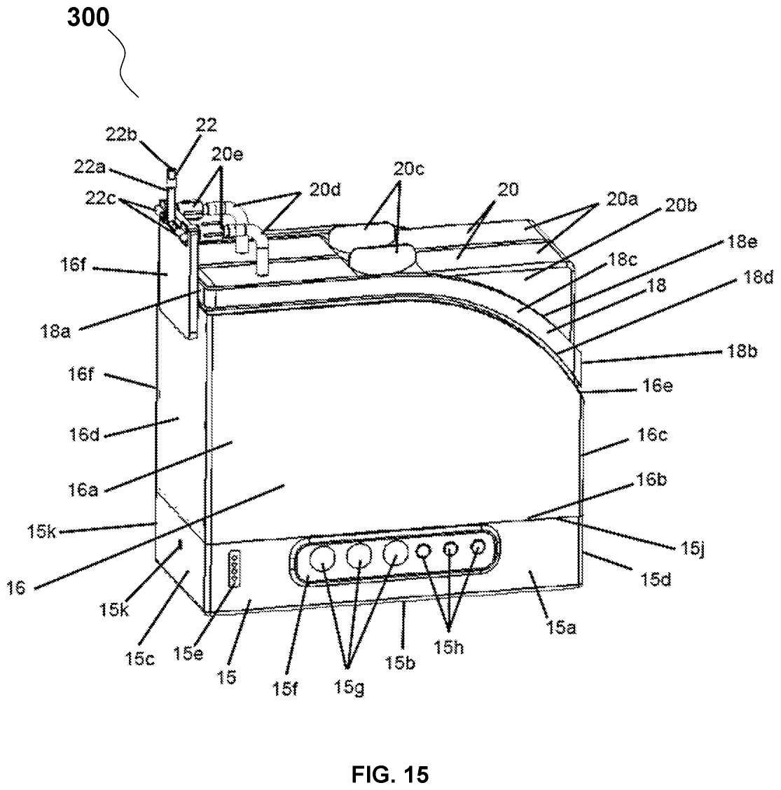

An example of a urine output monitoring system is shown in FIGS. 15 to 18. This system 300 comprises a base unit 15, a liner 18, bags in the shape of cartridges 20, and a connection tubing set 22. The base unit 15 contains core electronics, including the computing and sensing devices, for example, a scale for weighing fluid entering the cartridges 20, and circuitry displaying output data either on the base unit 15, remotely through a computer, or both. The displayed output can take many forms, including visual data, audible data, and tactile data. While the base unit 15 can function with only one cartridge 20, in an exemplary embodiment, there are at least two cartridges 20 that allow for constant flow and monitoring when one cartridge 20 is full and needs to be replaced.

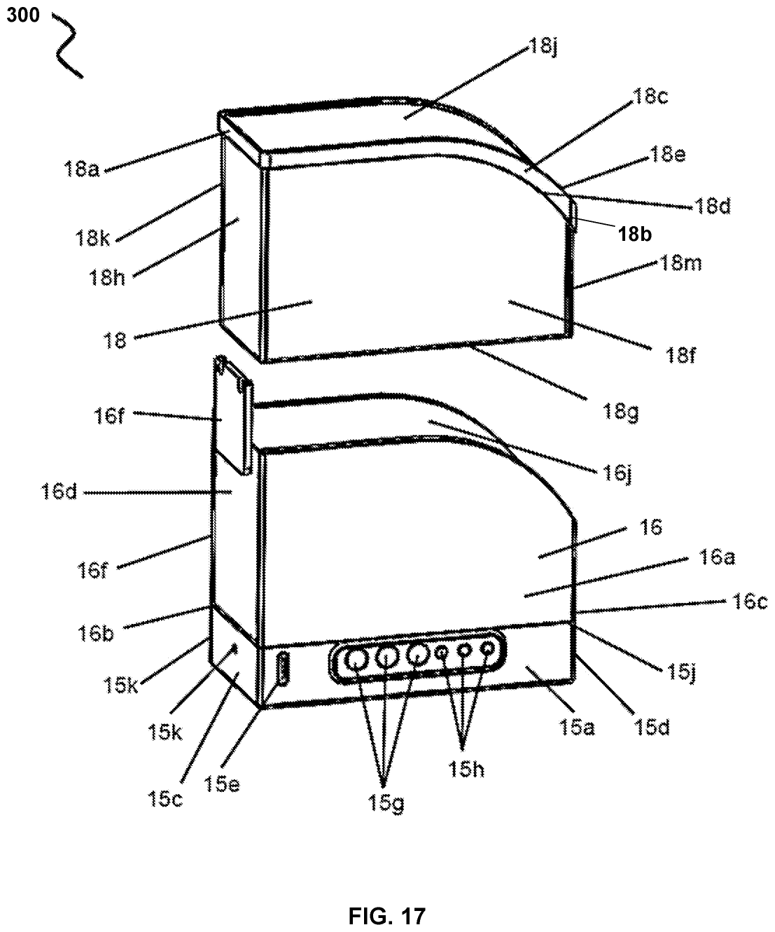

Shown in FIG. 15 is the lower section of the base unit 15, which contains the electronics or at least some portion thereof. The base unit 15 has a front face 15a, a bottom 15b, a first side 15c, a second side 15d opposite the first side 15c, a top 15j, and a back 15k. Also present is a battery level indicator 15e, an indicator panel 15f having indicator lights 15g and switches 15h. Attached to the base unit 15, for example, by plastic welding, adhesive, hardware, snap interfaces, or other approaches, is a center section 16. The center section 16 comprises a front face 16a, a lower face 16b, a first side 16c, a second side 16d opposite the first side 16c, a top face 16e, and an extension 16f. When the base unit 15 and the center section 16 are connected together, they form a body 17 of the system 300. With reference to FIG. 17, the liner 18 fits within center section 16 and comprises a first side lip face 18a, a second side lip face 18b opposite the first side lip face 18a, a front lip face 18c, a lower lip edge 18d, and top edge 18e. When placed inside the center section 16, the lower lip edge 18d of the liner 18 rests on the top face 16e of the center section 16 to limit the travel of the liner 18 into the hollow inner pocket 16j of the center section 16.

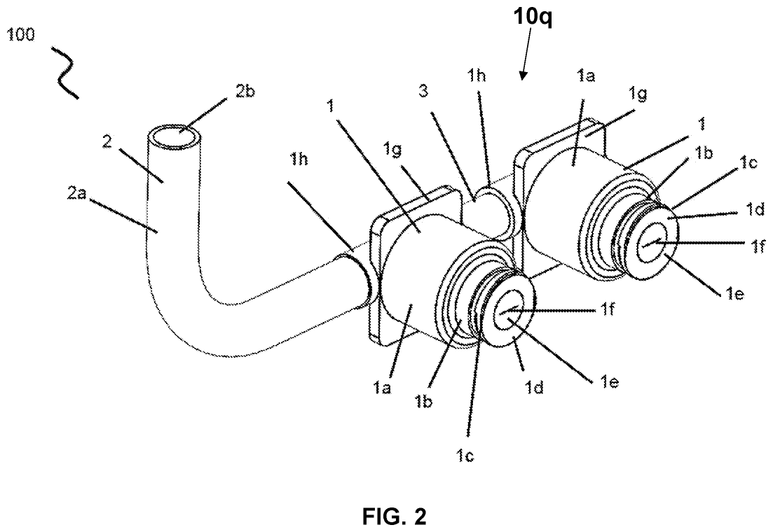

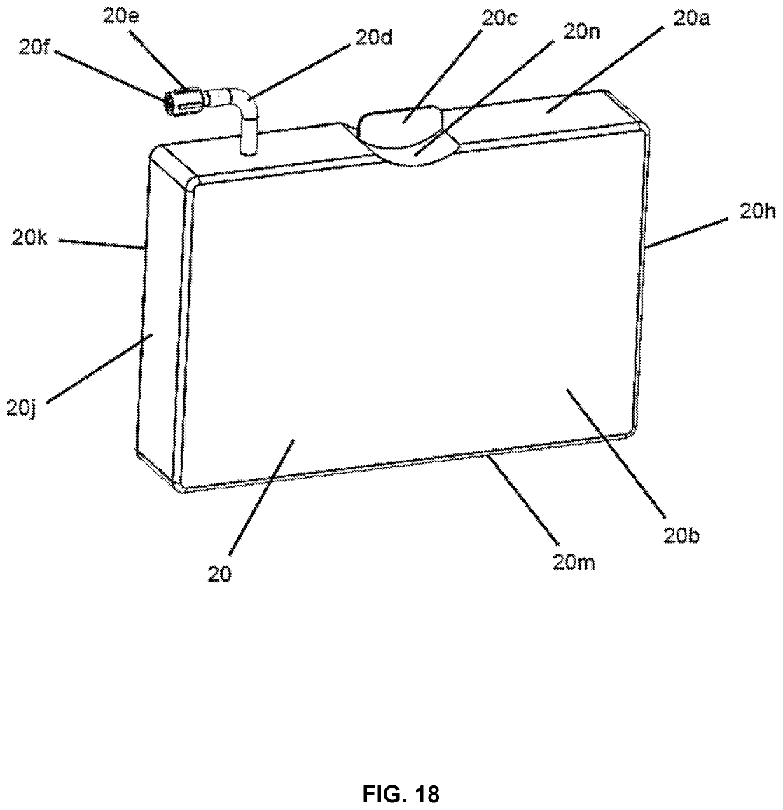

With reference to FIGS. 15 and 18, a cartridge 20 comprises a top face 20a, a side face 20b, one or more pull tabs 20c, at least one tube 20d that permits fluid flow into the cartridge 20, and a valve 20e connected to the tube 20d. A tubing set 22 shown in FIG. 15 is nearly identical to the embodiment shown and described in FIG. 2. In this exemplary configuration, a center tube 22a with an opening 22b feeds the two valves 22c. The tubing set 22 is supported and retained in a center section extension 16f and is removable to allow easy replacement.

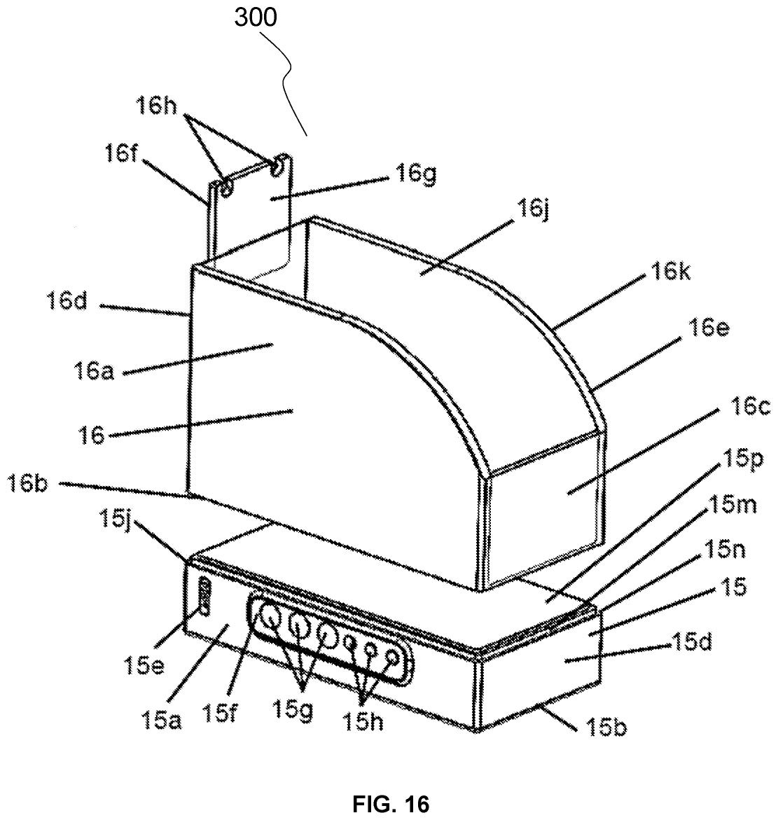

FIG. 16 shows more details of the base unit 15 and the center section 16. These sections can be machined or molded individually and then assembled by various techniques. The lower base unit 15 in this embodiment includes a lip 15m, which creates a ledge 15n against which the center section stops. In this embodiment, the top surface 15p is a plate attached to a non-illustrated weighing scale within the lower base unit 15. The center section 16 is shaped and configured to allow the liner 18 to fit therein in an easily insertable and smooth sliding motion to rest against the plate of the top surface 15p. The extension 16f has an inside face 16g and holders 16h that retain at least a portion of the tubing set 22. In this embodiment, the holders 16h are cylindrical and allow the valves 1 to snap into place and be temporarily retained therein. It is noted that the position of the ends of the valves 1 facing the cartridges 20 are out of the way sufficiently to allow for the cartridge 20 to be swapped out without hitting the valves 1. An inner pocket 16j allows the liner 18 to fit therein. If a liner 18 is not used, at least one cartridge 20 and, in an exemplary embodiment, two cartridges 20 fit within the center section 16. The back of the center section 16 is shown as 16k in FIG. 16.

With regard to FIG. 17, while a liner 18 is not necessary for the system 100, 200, 300 to function, it is another feature that allows for easier maintenance and cleanliness. In particular, the liner 18 protects the base unit 15 from contamination and can be disposed of and replaced as needed. The liner 18, in addition the previously disclosed features, has a front face 18f, a back face 18k opposite the front face 18f, a bottom face 18g, a first side face 18h, a second side face 18m opposite the first side face 18h, and a pocket 18j. The pocket 18j is large enough to accept the cartridges 20 or bags 6, 7 therein. The liner 18 freely slides within the center section 16 to allow for the weight of the cartridges 20 or the bags 6, 7 to be read accurately by the scale under the top surface 15p.

The cartridge 20, which can have rigid, semi-rigid, or flexible sides, is in an exemplary embodiment made from a recyclable plastic. The cartridge 20, in addition to the previously explained features, has a first side 20h, second side, 20j opposite the first side 20h, a back face 20k, and a lower face 20m. The top face defines a finger recess 20n adjacent the pull tab(s) 20c for ease of gripping. The valve 1 has an external sleeve that rotates to screw onto the tubing set 22, and an opening 20f that allows fluid to pass in and through the valve 1. There are, of course, variations to this configuration that can be altered. While it is preferred to have a check or one way valve, tubing from the cartridge could be connected to an input tube directly. Also, the cartridge can have a different look, shape, or form. Pull tab 20c can be replaced with a handle or a feature on the side faces 20b, 20k of the cartridge 20 to allow for easy grabbing for insertion and removal, in which case, the finger recess 20n is removed.

In addition, the base unit 15 shown in FIGS. 15 to 17 can have lights and buttons replaced by one or more LED or LCD displays. This display(s) may also be a touch screen. In that configuration, data is displayed directly on the base unit 15, with or without a remote receiver to display that data. In summary, the base unit 15 has some or all of the user interfaces, including items such as buttons, lights, touchscreen or screen, or any combination thereof.

When the base unit 15 is active, the cartridges 20 or bags 6, 7 placed within the center section 16 on the scale are effectively tared. This function compensates for weight of the cartridges and removes it out of the weighing equation. By using an accurate scale, a change in weight of the cartridges 20 or bags 6, 7 can be carefully monitored and tracked. When a cartridge 20 or bag 6, 7 is full, it is, therefore, possible to indicate this fact to a user, for example, when a preset weight is reached. It is possible to also use an alternative detector, such as light transmission, to determine a level of fluid, or to use a mechanical float. However, weight is a reliable unit and a scale in the base unit 15 reduces the number of components needed in the system 100, 200, 300.

When a cartridge 20 or bag 6, 7 is full and removed, in a desirable embodiment, manually or automatically a removal trigger is actuated to cause a brief pause in data collection. Removing the cartridge 20 may cause a spike in the data collection that is not relevant to the patient's data and may create a misleading change in how the data is interpreted. By pausing data collection, this spike is eliminated. When a new empty cartridge 20 replaces the removed cartridge 20, data can resume, and any urine collected in the brief change period can be accounted for, as a two cartridge system prevents loss of urine.

While it is desirable to have one weight scale under both cartridges 20, in an exemplary embodiment where individual cartridge data is unimportant, multiple scales, one for each cartridge, can be supplied and date combined from both scales.

To connect the cartridges 20 to the fluid input, the tubing set 22 allows the urine to flow to both cartridges 20. As discussed herein, a set of valves 1 allow for the cartridges 20 to be connected and disconnected without loss of fluid or urine. In addition, these self-sealing valves 1 reduce risk of contaminants entering the system 100, 200, 300 when a valve 1 is disconnected. By making the tubing set 22 sterile and disposable, along with the cartridges 20 or bags 6,7, the system 100, 200, 300 reduces any risk of contamination or infection.

In a desirable embodiment, the base unit 15 allows and compensates for a liner 18 in which the cartridges 20 rest. This adds additional cleanliness to the base unit 15 as an inexpensive liner 18 can be easily replaced. The cartridges 20 fit with the liner 18. The liner 18 is free to slide within the base unit 15 to allow the liner 18 to exert weight against the scale or scales located in the base unit 15. The liners 18 can be calibrated for weight or simply be tared when the unit is turned on or by manually pushing a button or telling a remote application that the liner 18 has been changed.

Digital scales are well understood in the art and examples use strain gauges or a load cell to convert force into an electrical signal. One or more strain gauges are placed under the load, and the amount of compression of the strain gauge or gauges affects electrical resistance of the strain gauge. The electrical resistance changes current flow, and the current change reflects force exerted by the load. Such an approach is extremely accurate. A signal conditioner then converts the input from the load cell into usable weight data.

By tracking a change in weight over time, the volume of urine or fluid entering the system 100, 200, 300 can be easily tracked. Water weighs 1 gram per milliliter. Normal urine weighs approximately 1.02 grams per milliliter. Appropriate software in the circuits of the base unit 15 can easily calculate the weight, divide it by 1.02, and provide a figure for volume. As the desired data has a general baseline and changes over time, any small error in actual urine weight will be negligible. However, in an exemplary embodiment, as part of the software, it is possible to input direct data from a lab test to compensate and provide higher accuracy, if needed. These features also add a desirable characteristic if the system 100, 200, 300 is used for an application or fluid other than urine, whereby the weight of the alternate fluid can be input into the software and data calculated off this new weight.

The data output from the base unit 15 can be transmitted by Bluetooth®, wireless transmission of data, or both to a remote, non-illustrated receiver. The data can be sent to a remote monitor screen, which may include a smart pad (e.g., iPad), a smartphone, or an equivalent. As the data transmitted is patient-specific and tampering with the data could negatively affect the data and treatment of the patient, in a desirable embodiment, the data is encoded and/or encrypted.

It is expected that multiple units could be in operation at the same facility, and other equipment in the area will be also be operating on wireless or Bluetooth® transmission, each base unit 15 has its own identification (e.g., IP address). This is well understood in the art. Thus, it will be easy to track and separate data transmitted from each of the systems 100, 200, 300 used. Through software, or an App, the hospital, or medical or other facility, can connect and tie the data directly to a patient ID in a desirable manner.

By tracking the data from the system 100, 200, 300, or variations thereof, changes in fluid volume can be tracked over time. This data can be supplied raw, such as in direct milliliters per hour or over any desired time interval, or graphed over time to provide a more visual analysis of the data. The data or the graph provides direct analysis and evaluation of trends. For example, if a patient has stable fluid input and the graph shows a downward trend, with reducing urine input, it may indicate fluid retention, kidney issues, or other health problems based on the patient conditions. By having the trends immediately available and digitally recorded (and transmissible immediately to a health care provided when desirable), the results can be provided much faster and more accurately then the current manual technique of having to read levels on the bags 6, 7 and record the values in a patient's chart.

For patients in critical care, or other locations, data from the unit can also be sent digitally to a smartphone or smart pad of a medical care provider, who may be monitoring the patient remotely. This can be extremely helpful for specialists with multiple patients, allowing the health care provider an ability to see patients while having immediate access to the data, or to receive alerts that a negative trend is occurring.

In an optional embodiment, a limit or limits may be entered at particular set points into the app or program. For example, if a medical care provider is concerned about a fluid output level dropping below a certain point, a limit can be set to reflect this data point. Should this limit be reached, an alarm can be sent quickly.

In another embodiment, electronics are provided to determine a clarity and/or color of the fluid entering the cartridges 20. These measurements can also be part of the data package, either displayed on the base unit 15 and/or transmitted remotely.

In a further embodiment, flow measurement data can be provided, e.g., with a flow meter. Some flow meters have moving parts that rotate as fluid passes by, and the motion is used to calculate flow. Existing flow meters use current differentials to determine flow from point to point. These alternative flow measurement approaches can be substituted or added. It is noted that measuring weight is simpler and does not require any contact with the fluid.

For general use in a hospital, the system 100, 200, 300 can be mounted at a bedside. This is similar to the current manual approach and avoids any tubes from extending beyond the bed. With variations to the number and types of beds currently in use, the system 100, 200, 300 has a series of threaded connectors or adapters that allow the systems 100, 200, 300 to be mounted to the bed. Of course, the system 100, 200, 300 can be mounted to another structure, whether fixed or mobile.

For data back-up and movement of a hospital bed to which the system 100, 200, 300 is attached, the system 100, 200, 300 has a built-in battery, e.g., in the base unit 15, that is rechargeable and/or replaceable. While replaceable and rechargeable battery packs eliminate a need for plugging the unit in at all, it is also desirable to have a mains cord that allows the system 100, 200, 300 to be plugged in during use where movement is not typical, expected, or often. In such a configuration, a battery life display indicates when the battery is low and needs charging and/or replacement. The battery can be recharged when the system 100, 200, 300 is reconnected to a wall power mains, for example.

It is preferred that the cartridges 20, bags 6, 7, liner 18, and/or tubing set 22 be recyclable. As such, the cartridges 20 can have a port to allow a user to empty the fluid therein prior to disposal.

In addition, for attachment to a structure such as a bed, the system 100, 200, 300 has a thickness, size, or distance that allows the system 100, 200, 300 to innocuously attach thereto, i.e., it is smaller than the structure to which it attaches. This will reduce any risk that the system 100, 200, 300 interferes with personnel or be contacted, hit, or damaged.

In general, in an exemplary embodiment, the base unit 15, the liner 18, and the cartridges 20 will be molded from plastic. However, other materials and methods can be used, such as metals, elastomers for the cartridges 20 or bags 6, 7, or 3D-printing and/or machining of one or more sections of the system 100, 200, 300.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The foregoing description and accompanying drawings illustrate the principles, exemplary embodiments, and modes of operation of the systems, apparatuses, and methods. However, the systems, apparatuses, and methods should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the systems, apparatuses, and methods as defined by the following claims.

The invention has been described in an illustrative manner, and it is to be understood that the terminology, which has been used is intended to be in the nature of words of description rather than of limitation. Many modifications and variations of the present systems, apparatuses, and methods are possible in light of the above teachings. It is, therefore, to be understood that within the scope of the appended claims, the invention can be practiced otherwise than as specifically described.

What is claimed is:

1. A fluid collection system, comprising:
a hollow tube comprising:
a tube input configured to connect removably to a fluid supply tube and, responsive to connection, to receive input fluid therethrough; and
a tube output configured to output the input fluid;
a first valve having flow-on and flow-off states and comprising:
a first input configured to regulate an amount of flow through the first valve; and
a first output;
a second valve having flow-on and flow-off states and comprising:
a second input configured to regulate an amount of flow through the second valve; and
a second output;
the first and second valves each:
fluidically connected to the tube output;
configured to receive the input fluid respectively; and
configured to output the input fluid from the hollow tube when in the flow-on state; and
at least one collection container comprising:
a fluid bag defining a water-tight interior and comprising an input shaped to receive fluid therethrough and into the interior; and
a removable connector:
comprising an input configured to removably connect to one of the first and second outputs;
comprising an output fixed to the fluid bag and defining a lumen fluidically connected to the interior of the fluid bag; and
responsive to connection to the one of the first and second outputs, configured to receive fluid from the hollow tube through the one of the first and second outputs.

2. The system according to claim 1, further comprising a pressure control valve fluidically connected to the tube input, having a pressure flow setting, and being configured to permit flow of the input fluid into the tube input when the input fluid is at a pressure above the pressure flow setting.

3. The system according to claim 1, wherein the first and second valves have a steady state in the flow-off state and:
connection to the at least one collection container automatically places either of the first and second valves in the flow-on state; and
disconnection from the at least one collection container automatically returns the one of the first and second valves to the flow-off state.

4. The system according to claim 1, wherein the at least one collection container is a modular set of at least first and second collection containers each respectively connected to one of the first and second outputs to permit one of the at least first and second collection containers to be in either of the flow-on and flow-off states while the other of the at least first and second collection containers are in either of the flow-on and flow-off states.

5. The system according to claim 4, wherein the at least first and second collection containers are configured such that, responsive to the interior of one of the at least first and second collection containers being full, flow of the input fluid is directed to the other of the at least first and second collection containers.

6. The system according to claim 1, wherein the removable connector of the at least one collection container has a container valve configured to enter a fluid-closed state automatically when the interior is full of liquid.

7. The system according to claim 1, further comprising a fluid monitoring system connected to the hollow tube and configured:
to measure fluid output; and
to transmit data associated with the fluid output to a receiver to permit direct reading of the fluid output.

8. The system according to claim 7, wherein the fluid monitoring system has a defined measurement range for the fluid output and is configured to indicate if the measured fluid output falls at least one of:
within the defined measurement range; and
outside the defined measurement range.

9. The system according to claim 8, wherein the fluid monitoring system is configured to transmit and remotely create an alarm if the measured fluid output is outside the defined measurement range.

10. The system according to claim 1, further comprising a fluid monitoring system connected to the hollow tube and configured:
to measure at least one of fluid color and fluid transparency; and
to transmit data associated with the measurement of the at least one of fluid color and fluid transparency to a receiver to permit direct reading of the at least one of fluid color and fluid transparency.

11. The system according to claim 10, wherein the fluid monitoring system has a defined measurement range for the at least one of fluid color and fluid transparency and is configured to indicate if the at least one of the measured fluid color and the measured fluid transparency falls at least one of:
within the defined measurement range; and
outside the defined measurement range.

12. The system according to claim 11, wherein the fluid monitoring system is configured to transmit and remotely create an alarm if at least one of the measured fluid color and measured fluid transparency is outside the defined measurement range.

13. The system according to claim 1, further comprising a hospital bed connector configured to secure at least one of the hollow tube, the first and second valves, and the at least one collection container to a hospital bed.

14. The system according to claim 1, wherein the at least one collection container comprises a quantity of fluid-absorbing media within the interior.

15. The system according to claim 1, wherein the interior of the at least one collection container is subdivided into at least two fluid-receiving compartments.

16. A fluid collection system, comprising:
a hollow tube comprising:
a tube input configured to connect removably to a fluid supply tube and, responsive to connection, to receive input fluid therethrough; and
a tube output configured to output the input fluid;
a valve having flow-on and flow-off states and comprising:
a valve input configured to receive fluid flow therethrough; and
at least two outputs including a first output and a second output, the valve separating the valve input from the at least two outputs and directing flow independently from the valve input to each of the at least two outputs.

17. The system according to claim 16, wherein:
the valve comprises a sensor receiver configured to receive sensor data; and
the valve input directs fluid flow from the input to at least one of the two outputs at a time based upon received sensor data.

18. The system according to claim 17, further comprising:
at least one collection container fluidically connected to one of the at least two outputs; and
at least one sensor configured to read the at least one collection container and determine whether the at least one collection container is at least one of full and missing.

19. The system according to claim 17, further comprising:
a plurality of collection containers each comprising at least one sensor configured to determine if the respective one of the collection containers is at least one of full and missing, at least one of the plurality of collection containers fluidically connected to one of the at least two outputs.

20. A fluid collection system, comprising:
a hollow input tube configured to connect removably to a fluid supply tube supplying input fluid;
first and second valves separate from one another and each having flow-on and flow-off states, each of the first and second valves fluidically connected to the hollow input tube, respectively:
to receive the input fluid; and
to output the input fluid when in the flow-on state; and
at least one hollow collection container defining a water-tight interior and comprising a removable connector configured to removably connect to one of the first and second valves and, responsive to connection thereto, configured to receive the input fluid.
